(19) United States
(12) Patent Application Publication
(10) Pub. No.: US 2022/0176043 A1
(43) Pub. Date: Jun. 9, 2022

(54) GRIP FOR MOUNTING A CYSTOTOME TO A SYRINGE, AN ASSEMBLY THEREOF, AND A METHOD RELATED THERETO

(71) Applicant: Beaver-Visitec International (US), Inc., Waltham, MA (US)

(72) Inventor: Paula Y. Wang, Sudbury, MA (US)

(21) Appl. No.: 17/534,614

(22) Filed: Nov. 24, 2021

Related U.S. Application Data
(60) Provisional application No. 63/120,872, filed on Dec. 3, 2020.

(51) Int. Cl. A61M 5/31; A61M 5/315

(52) U.S. Cl. CPC A61M 5/3137; A61M 2210/0612; A61M 2205/586; A61M 5/31511

(57) ABSTRACT

In one aspect of the subject invention, a grip is provided for mounting to a syringe, the grip including: an elongated body extending along a longitudinal axis between first and second ends, the first end defining a female luer fitting configured for mounting onto the syringe, an inlet opening being defined in the female luer fitting, the second end defining a male luer fitting defining an outlet opening, and, a liquid passageway defined through the body between the inlet opening and the outlet opening to allow liquid flow therebetween. The body further defines an outer surface encircling the longitudinal axis, the outer surface being at least partially textured. Advantageously, with the subject invention, a grip is provided which is configured to both mount to a syringe and to mountingly receive a cystotome, in providing an enhanced gripping surface for a practitioner performing a capsulotomy.

### CROSS-REFERENCE TO RELATED APPLICATION

This application claims priority to U.S. Provisional Application No. 63/120,872, filed Dec. 3, 2020, the entire contents of which are incorporated by reference herein.

### FIELD OF THE INVENTION

The subject invention relates to syringe assemblies used in ophthalmic surgery, particularly, in performing capsulotomies.

### BACKGROUND OF THE INVENTION

Cystotomes are known in the prior art for incising the lens capsule of a patient's eye in performing a capsulotomy, e.g., during capsulorhexis., as shown in FIG. 1. Examples of commercially-available cystotomes are irrigating cystotomes sold by Beaver-Visitec International, Inc. of Waltham, Mass. A cystotome is typically provided with a plastic hub from which the cystotome extends distally. The distal end of the cystotome defines an opening with a radially-protruding sharpened edge for incising the patient's eye, e.g., incising the anterior capsule of the patient's eye in performing a capsulotomy, and, the hub includes a female luer fitting for mounting onto a standard syringe. In use, the syringe allows for introduction of irrigation fluid through the cystotome during a capsulotomy.

Typically, a practitioner grips the syringe to manipulate, e.g., rotate, the cystotome during a capsulotomy, often adjusting their grip especially for a continuous curvilinear capsulorhexis (CCC). Standard syringe barrels typically have smooth outer surfaces and a constant-diameter barrel, providing no grip enhancement or ergonomic optimization, since grip enhancement is not a factor in administering injections.

### SUMMARY OF THE INVENTION

In one aspect of the subject invention, a grip is provided for mounting to a syringe having a luer tip with a liquid outlet defined at a distal end thereof, the grip including: an elongated body extending along a longitudinal axis between first and second ends, the first end defining a female luer fitting configured for mounting onto the luer tip of the syringe, an inlet opening being defined in the female luer fitting formed to align with the liquid outlet of the luer tip with the female luer fitting mounted to the luer tip, the second end defining a male luer fitting defining an outlet opening at a distal end thereof, and, a liquid passageway defined through the body between the inlet opening and the outlet opening to allow liquid flow therebetween. The body further defines an outer surface encircling the longitudinal axis, the outer surface being at least partially textured. Advantageously, with the subject invention, a grip is provided which is configured to both mount to a syringe and to mountingly receive a cystotome, in providing an enhanced gripping surface for a practitioner performing a capsulotomy.

In a further aspect of the subject invention, a syringe assembly is provided for performing a capsulotomy on a patient's eye with a cystotome, the syringe assembly including: a syringe having a luer tip with a liquid outlet defined at a distal end thereof; a grip having an elongated body extending along a longitudinal axis between first and second ends, the first end defining a female luer fitting configured for mounting onto the luer tip of the syringe, an inlet opening being defined in the female luer fitting formed to align with the liquid outlet of the luer tip with the female luer fitting mounted to the luer tip, the second end defining a male luer fitting defining an outlet opening at a distal end thereof, and, a liquid passageway defined through the body between the inlet opening and the outlet opening to allow liquid flow therebetween, wherein the body defines an outer surface encircling the longitudinal axis, the outer surface being at least partially textured, and, wherein the grip is mounted to the syringe with the female luer fitting being mounted to the luer tip of the syringe; and, a cystotome extending distally from a hub, the cystotome configured for incising the lens capsule of the patient's eye, the hub having a secondary female luer fitting mounted to the male luer fitting of the grip.

In yet a further aspect of the subject invention, a method is provided for performing a capsulotomy on a patient's eye, the method including: providing a grip having an elongated body extending along a longitudinal axis between first and second ends, the first end defining a female luer fitting with an inlet opening defined therein, the second end defining a male luer fitting defining an outlet opening at a distal end thereof, and, a liquid passageway defined through the body between the inlet opening and the outlet opening to allow liquid flow therebetween, wherein, the body defines an outer surface encircling the longitudinal axis, the outer surface being at least partially textured; mounting the female luer fitting of the grip onto a luer tip of a syringe containing irrigation fluid; and, mounting a hub, from which distally extends a cystotome, onto the male luer fitting of the grip.

As used herein, the term “distal,” and derivatives thereof (e.g., distally) refers to a direction towards or closer to a patient. As used herein, the term “proximal,” and derivates thereof (e.g., proximally) refers to a direction away or farther from a patient.

These and other features of the subject invention will be better understood through a study of the following detailed description.

### BRIEF DESCRIPTION OF THE DRAWINGS

FIG. 1 depicts the use of a cystotome in performing a capsulotomy, the cystotome being mounted to a standard syringe as known in the prior art;

FIG. 2 is a perspective view of a syringe assembly in accordance with the subject invention;

FIG. 3 is side view of the syringe assembly of FIG. 2;

FIG. 4 is an exploded view of syringe assembly of FIG. 2;

FIG. 5 is a cross-sectional view of a syringe useable with the subject invention; and,

FIGS. 6-14 are various views of grips useable with the subject invention.

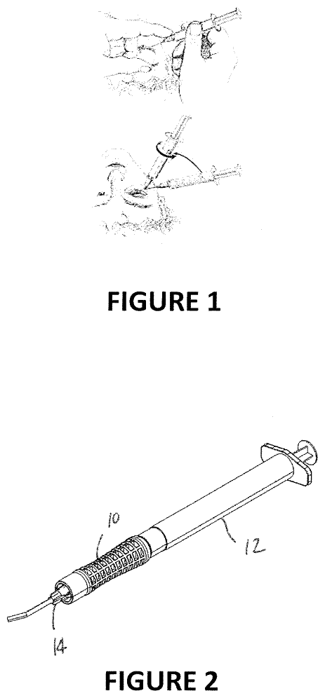

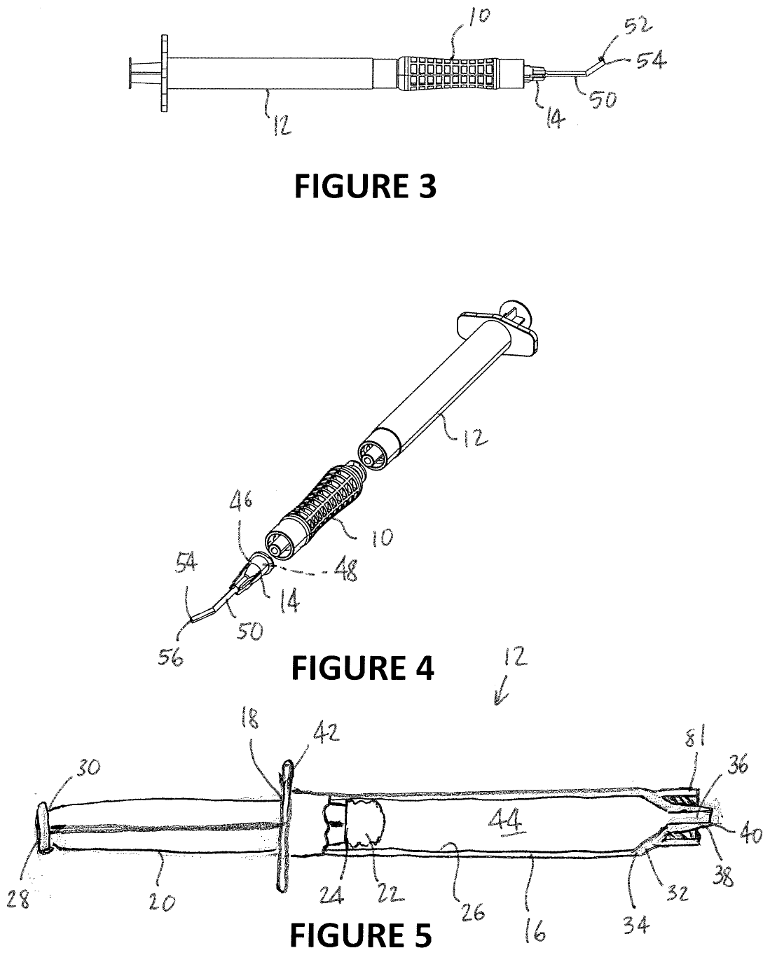

### DETAILED DESCRIPTION OF THE INVENTION

With reference to the Figures, a grip 10 is shown in accordance with the subject invention. The grip 10 is mountable to a syringe 12 and is configured to mountingly receive a cystotome 14.

As shown in FIG. 5, the syringe 12 may be any standard syringe, including being pre-fillable (e.g., glass barrel) or disposable (e.g., plastic barrel). The syringe 12 may include a barrel 16, formed of glass and/or plastic, for accommodating a liquid, which has an open proximal end 18, through which plunger 20 may extend. A piston 22, which may be elastomeric, may be provided at a distal end 24 of the plunger 20, the piston 22 formed for sliding, liquid-tight engagement with inner surface 26 of the barrel 16. A thumb pad 28 may be provided at a proximal end 30 of the plunger 20. A wall 32 may extend across a distal end 34 of the barrel 16. The wall 32 may be tapered to direct flow to a liquid outlet 36. The wall 32 may be configured to define a luer tip 38, as is known in the art. The luer tip 38 may be a slip tip or provided with a surrounding internally-threaded collar to provide a “luer lock” arrangement. The liquid outlet 36 extends through a distal end 40 of the luer tip 38. For ease of handling, one or more finger flanges 42 may protrude radially outwardly from the barrel 16 about the open proximal end 18.

The barrel 16 defines a reservoir 44 distally of the plunger 20. Distal movement of the plunger 20, results in distal movement of the piston 22 and, thus, liquid contained in the reservoir 44 being urged through the liquid outlet 36.

The cystotome 14 may be of any known type. By way of non-limiting example, the cystotome 14 may include a hub 46, which may be plastic, having a secondary female luer fitting 48 defined therein. The cystotome 14 may be in the form of a cannula 50, which may be bent, and secured to the hub 46 to extend distally therefrom. The cystotome 14 may be configured for incising a lens capsule of a patient's eye, e.g., by including one or more radially protruding cutting edge(s) 52 at or proximate to a distal end 54 of the cannula 50. The cystotome 14 may include a distal opening 56 formed at the distal end 54, through which irrigation fluid may flow. As is known in the art, a cap may be provided mounted to external portions of the hub 46 with an opening through which the cystotome 14 may extend.

The grip 10 includes a body 58, which may be a unitary piece, prepared by various techniques e.g., by molding or forming (e.g., three-dimensional printing). The grip 10 may be formed of thermoplastic and/or elastomeric material. Material must be selected to allow the grip 10 to be sterilized (e.g., by UV exposure, gas exposure, steam exposure, and so forth).

The body 58 is elongated to extend along a longitudinal axis LA between first and second ends 60, 62 thereof. The first end 60 defines a female luer fitting 64 configured for mounting onto the luer tip 38 of the syringe 12. The female luer fitting 64 defines an inlet opening 66, which is formed to align with the liquid outlet 36 of the luer tip 38 with the female luer fitting 64 being mounted to the luer tip 38. The second end 62 defines a male luer fitting 68 which defines an outlet opening 70 at a distal end 72 thereof. A liquid passageway 74 may be defined through the body 58 between the inlet opening 66 and the outlet opening 70 to allow liquid flow therebetween. The liquid passageway 74 preferably is straight, to run along the longitudinal axis LA. Changes in direction are not desired since such may cause resistance to flow through the liquid passageway 74. It is also preferred that the liquid passageway 74 generally have a constant cross-section. The liquid passageway 74 may taper in a direction from the inlet opening 66 to the outlet opening 70, preferably, the taper being gradual.

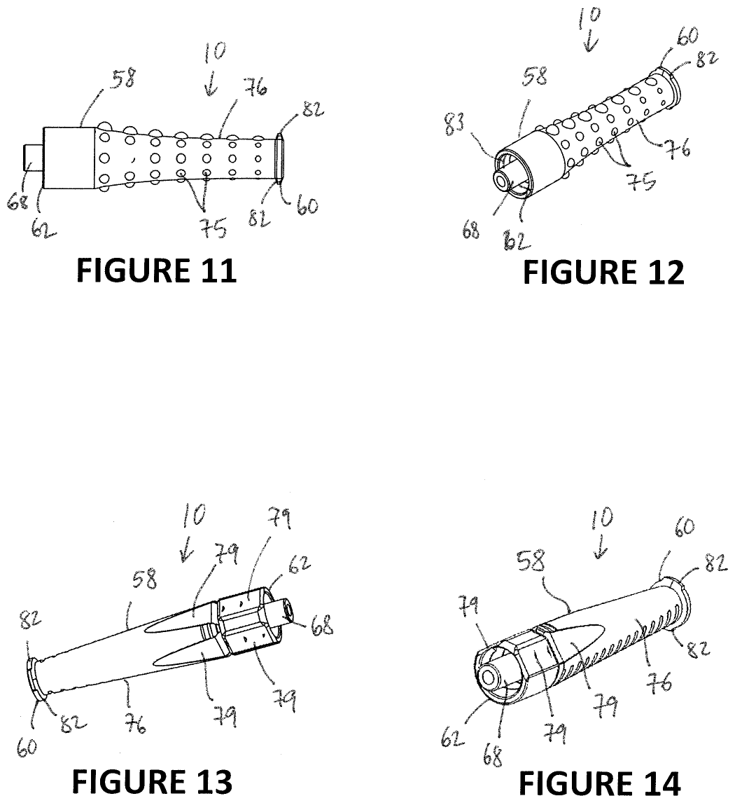

The body 58 defines an outer surface 76 which encircles the longitudinal axis LA. The outer surface 76 is at least partially textured, to enhance grip therewith. A full circumference of the body 58 may be textured, e.g., to define an annular band of texturing, to provide a practitioner with a radially uninterrupted textured surface for rotational adjustment of the cystotome 14. The texturing may be provided by any known arrangement, including, but not limited to, raised elements 75 (such as beads and/or ribs) and/or hollows 77 (such as channels or dimples). FIGS. 11 and 12 show exemplary forms of the raised elements 75, particularly as raised beads. The raised elements 75 may include regular or irregular patterns of protruding or embossed elements. Likewise, the hollows 77 may include regular or irregular patterns of recesses or indentations. For example, as shown in FIG. 7, the hollows 77 may include spaced-apart discontinuities 78 defined in the outer surface 76 where open pockets are defined between solid portions of the outer surface 76. The discontinuities 78 may be defined by a plurality of intersecting ribs 80. In addition, or alternatively, the outer surface 76 may be defined by an anti-slip material which provides enhanced gripping. Examples of suitable anti-slip materials include one or more of a polymeric foam, a polymeric mesh, an elastomer, and a coated or treated polymer. The body 58 may be provided as a two-part piece with a core of one material encased by the outer surface 76 of a different material. In this configuration, the core may be formed as a unitary piece with the outer surface 76 being applied thereto as a secondary operation. The core may contain the female luer fitting 64, the male luer fitting 68, and the liquid passageway 74.

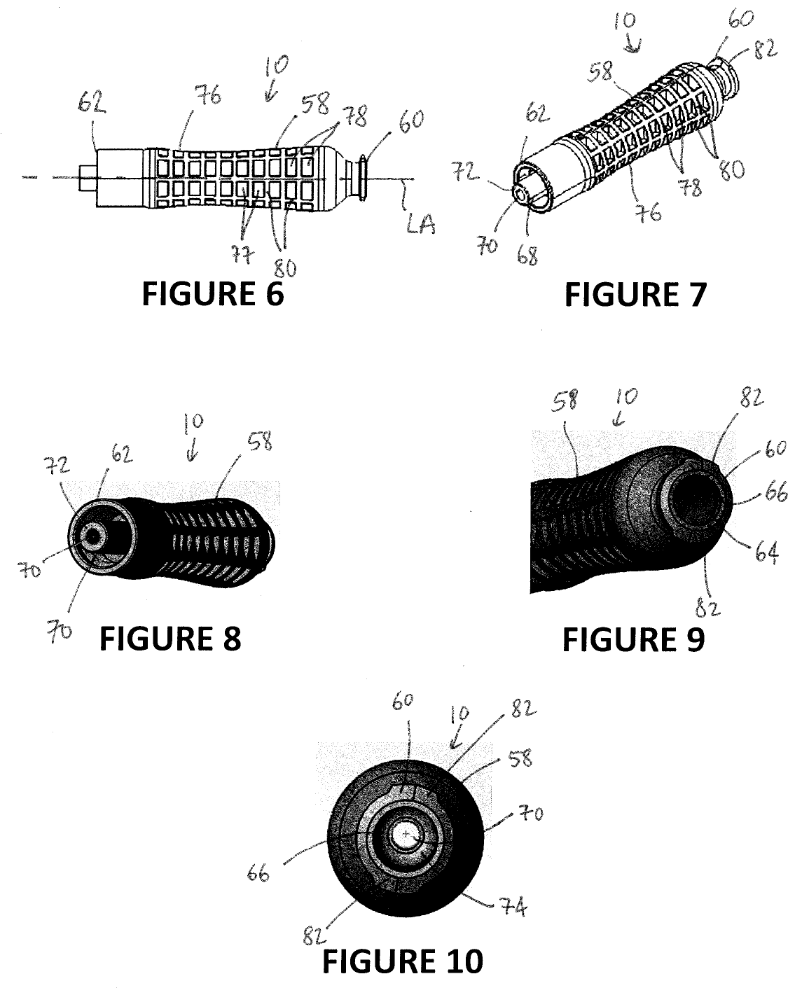

In addition, or alternatively, the body 58 may be configured to enhance handling. For example, as shown in FIG. 6, the diameter of the body 58 may vary along the longitudinal axis LA. This allows for the body 58 to have a concave shape, with indentations in which portions of a practitioner's fingers may nest. Other shapes are possible such as one or more undulations, convexities, and so forth. In addition, as shown in FIGS. 13-14, portions of the body 58 may be flattened or otherwise configured to define one or more panels or truncations 79.

The grip 10 may be mounted to the syringe 12 with the female luer fitting 64 being mounted to the luer tip 38. One or more thread elements 82 may be provided at the first end 60, e.g., about the female luer fitting 64, configured to engage, e.g., threadedly engage, a threaded collar 81 if provided about the luer tip 38 to form a “luer lock.” Slip mounting alone may be utilized. In addition, the cystotome 14 may be mounted to the grip 10 with the secondary female luer fitting 48 being mounted to the male luer fitting 68. An internally-threaded collar 83 may be provided about the male luer fitting 68, as shown in FIG. 12, with one or more cooperating thread elements provided on the hub 46, about the secondary female luer fitting 48, to allow a “luer lock” to form therebetween. Slip mounting alone may be also utilized at this interface.

The grip 10, the syringe 12, and the cystotome 14 collectively form a syringe assembly useable for performing a capsulotomy on a patient's eye. The grip 10 provides a practitioner with an enhanced grip surface distally of the syringe 12, to allow for good handling. Many practitioners use a rolling motion of a cystotome in performing a capsulotomy. With the outer surface 76 being textured, the practitioner is provided with an improved grip surface. With the outer surface 76 being textured about its entire circumference, an improved grip surface may be provided agnostic to rotational orientation of the grip 10.

In use, an assembly for performing a capsulotomy on a patient's eye may be prepared by providing the grip 10. The grip 10 is required to be sterilized for use, as well as, the syringe 12 and the cystotome 14. The grip 10 is mounted to the syringe 12 and the cystotome 14 is mounted to the grip 10, e.g., via the hub 46. With this arrangement, the syringe 12 may be provided with irrigation fluid that may be dispensed through the cystotome 14 during a capsulotomy.

What is claimed is:

1. A grip for mounting to a syringe having a luer tip with a liquid outlet defined at a distal end thereof, the grip comprising:
an elongated body extending along a longitudinal axis between first and second ends, the first end defining a female luer fitting configured for mounting onto the luer tip of the syringe, an inlet opening being defined in the female luer fitting formed to align with the liquid outlet of the luer tip with the female luer fitting mounted to the luer tip, the second end defining a male luer fitting defining an outlet opening at a distal end thereof, and, a liquid passageway defined through the body between the inlet opening and the outlet opening to allow liquid flow therebetween, wherein, the body defines an outer surface encircling the longitudinal axis, the outer surface being at least partially textured.

2. The grip of claim 1, wherein the diameter of the body varies along the longitudinal axis.

3. The grip of claim 2, wherein the body defines a concave shape.

4. The grip of claim 1, wherein the outer surface is textured by a plurality of spaced-apart discontinuities and/or raised elements.

5. The grip of claim 4, wherein the outer surface includes a plurality of intersecting ribs defining the spaced-apart discontinuities.

6. The grip of claim 1, further comprising an internally-threaded collar disposed about the male luer fitting.

7. The grip of claim 1, further comprising at least one thread protruding radially outwardly from the female luer fitting.

8. The grip of claim 1, wherein the outer surface is textured about a full circumference of the body.

9. A syringe assembly for performing a capsulotomy on a patient's eye with a cystotome, the syringe assembly comprising:
a syringe having a luer tip with a liquid outlet defined at a distal end thereof;
a grip as in claim 1 with the female luer fitting thereof mounted to the luer tip of the syringe;
and,
a cystotome, distally extending from a hub, the cystotome configured for incising the lens capsule of the patient's eye, the hub having a secondary female luer fitting mounted to the male luer fitting of the grip.

10. A method of preparing an assembly for performing a capsulotomy on a patient's eye, the method comprising:
providing a grip having an elongated body extending along a longitudinal axis between first and second ends, the first end defining a female luer fitting with an inlet opening defined therein, the second end defining a male luer fitting defining an outlet opening at a distal end thereof, and, a liquid passageway defined through the body between the inlet opening and the outlet opening to allow liquid flow therebetween, wherein, the body defines an outer surface encircling the longitudinal axis, the outer surface being at least partially textured;
mounting the female luer fitting of the grip onto a luer tip of a syringe containing irrigation fluid; and,
mounting a hub, from which distally extends a cystotome, onto the male luer fitting of the grip.

11. The method of claim 10, wherein the outer surface is textured about a full circumference of the body.
